(19) United States
(12) Patent Application Publication
(10) Pub. No.: US 2005/0260238 A1
(43) Pub. Date: Nov. 24, 2005

(54) Cosmetic compositions comprising wholly hydrophilic block copolymers and particular anti-fungal agents

(75) Inventors: Franck Giroud, Clichy (FR); Lydia Panangatte, Poissy (FR); Claude Dubief, Le Chesnay (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: L'OREAL, PARIS (FR)

(21) Appl. No.: 11/123,110

(22) Filed: May 6, 2005

Related U.S. Application Data
(60) Provisional application No. 60/587,027, filed on Jul. 13, 2004.

(30) Foreign Application Priority Data
May 13, 2004 (FR) 04/50932

(51) Int. Cl. A61K 31/555; A61K 7/00

(52) U.S. Cl. 424/401; 514/184

(57) ABSTRACT

Topically applicable cosmetic/dermatological compositions useful for treating dandruff and/or squamae of the human scalp contain at least one anti-fungal agent selected from the group consisting of pyridinethione salts, 1-hydroxy-2-pyrrolidone compounds, 2,2′-dithiobis(pyridine N-oxide) and selenium sulfides, and mixtures thereof, and at least one wholly hydrophilic block copolymer which comprises at least one polyionic block, formulated into a topically applicable, physiologically acceptable medium therefor.

### CROSS-REFERENCE TO PRIORITY/PROVISIONAL APPLICATIONS

This application claims priority under 35 U.S.C. § 119 of FR 04/50932, filed May 13, 2004, and of provisional application Ser. No. 60/587,027, filed Jul. 13, 2004, each hereby expressly incorporated by reference and each assigned to the assignee hereof.

### BACKGROUND OF THE INVENTION

1. Technical Field of the Invention

The present invention relates to cosmetic compositions in particular for treating dandruff and squamae of the scalp, comprising at least one anti-fungal agent selected from among pyridinethione salts, 1-hydroxy-2-pyrrolidone compounds, 2,2′-dithiobis(pyridine N-oxide), selenium sulfides and mixtures thereof, and at least one block copolymer exclusively containing hydrophilic blocks and comprising at least one polyionic block.

The present invention also relates to an anti-dandruff treatment regime or regimen which entails applying the above compositions to the scalp and then rinsing the scalp.

The subject compositions have improved anti-fungal properties and can combat dandruff and squamae of the scalp more efficiently. Same are also effective on sensitive and reactive scalps and furthermore impart to the hair excellent cosmetic properties, such as a pleasant feel and satisfactory sheen and lightness.

This invention more especially relates to anti-dandruff shampoos.

2. Description of Background and/or Related and/or Prior Art

It is known practice to use as anti-dandruff active agents products that inhibit microbial proliferation and/or that have anti-fungal properties, for instance pyridinethione salts, 1-hydroxy-2-pyrrolidone derivatives or selenium sulfides, in order to combat the formation of dandruff or squamae on the scalp, this formation generally being accompanied by microbial and/or fungal proliferation. However, these anti-dandruff active agents often have insufficient activity, necessitating long and repeated use of these products in order for the user to first experience the benefits thereof. This often results in early abandonment of the treatment by the users.

Thus, need continues to exist for an effective anti-dandruff product with prolonged and rapid action.

Aqueous biocidal compositions for the biocidal treatment of textile or cutaneous keratin surfaces, or industrial or domestic hard surfaces, are described in WO-A-00/02449 to Rhodia. These compositions contain a biocidal agent, a surfactant when the biocide is hydrophobic, and a water-soluble or water-dispersible organic copolymer comprising at least one macromolecular or oligomeric unit that can interact with the biocide or the micelles of the surfactant containing the said biocide if the latter is hydrophobic, and a hydrophilic macromolecular unit that can interact with the surface to be treated and possibly with the biocide.

A biologically active composition comprising a supramolecular complex including a block copolymer, containing at least one water-soluble nonionic segment and at least one polyionic segment, and of an ionic surfactant containing hydrophobic groups, is moreover described in WO-A-98/56334 to the University of Nebraska; the ionic charge of the surfactant is opposite that of the polyionic segment of the block copolymer and the constituents of the complex are linked together by interaction between the opposite charges and the hydrophobic groups. In addition, when this complex contains an anionic surfactant with biological activity, then this anionic surfactant has a net charge of less than or equal to 10. This composition may also contain anti-fungal agents such as azoles or macrolides and compounds such as sorbic acid, ethylenediaminetetraacetic acid or benzalkonium chloride.

A washing composition, in particular a household composition for washing greasy articles such as kitchen crockery and cookware, containing cationic block copolymers is also described in WO-A-00/71591 to Rhodia. This composition may optionally contain a large number of additives, including enzymes, nacreous agents, anti-fungal agents, fragrances, preservatives, silicones and surfactants.

A cosmetic composition comprising a diblock copolymer A-B such that: block A is polycationic at the pH of the composition; block B is neutral at the pH of the composition and blocks A and B both contain units derived from an unsaturated α-ethylenic monomer, is also described in WO-A-03/050 184 to Rhodia. These copolymers are intended to improve the deposition of the other ingredients of the composition, for instance silicones or cationic polymers, on the hair.

Moreover, a composition containing: a polymer that is polyionic at the pH of the composition, which may be polycationic or polyanionic; a compound that is ionic at the pH of the composition; a copolymer comprising at least a first portion that is polyionic at the pH of the composition, which is polycationic if the ionic compound is anionic, or polyanionic if the ionic compound is cationic, and a second portion that is neutral at the pH of the formulation; and also at least one of these portions contains units derived from unsaturated mono-α-ethylenic monomers, is moreover described in WO-A-03/050 185 to Rhodia. The ionic compound is preferably a surfactant.

Hairwashing compositions containing, in a cosmetically acceptable aqueous or aqueous-alcoholic medium, an anionic or nonionic linear block copolymer, comprising a hydrophobic block and a hydrophilic block, at least one anionic surfactant combined with a nonionic surfactant and/or an amphoteric surfactant and, respectively, at least one cationic or amphoteric polymer or a silicone which is non-volatile at room temperature, are also described in FR-A-2,840,205 and FR-A-2,840,206 to L'Oréal. These compositions may optionally contain a large number of cosmetic ingredients, including preservatives and anti-dandruff agents.

Aqueous hair treatment compositions containing block copolymers comprising at least one polyionic block and at least one neutral block, which have good affinity for the hair, are also described in WO 03/050195.

None of these prior art documents mentions or suggests the technical problem of reinforcing the activity of certain anti-fungal agents against dandruff and squamae of the scalp.

### SUMMARY OF THE INVENTION

It has now surprisingly been found that it is possible to considerably reinforce the anti-dandruff activity of certain anti-fungal agents by combining them with block copolymers exclusively consisting of hydrophilic blocks and comprising at least one polyionic block.

Thus, the present invention features compositions containing a physiologically acceptable medium, at least one anti-fungal agent selected from among pyridinethione salts, 1-hydroxy-2-pyrrolidone compounds, 2,2′-dithiobis(pyridine N-oxide) and selenium sulfides, and mixtures thereof, and at least one block copolymer exclusively containing hydrophilic blocks and comprising at least one polyionic block.

This invention also features the cosmetic administration of at least one block copolymer exclusively containing hydrophilic blocks and comprising at least one polyionic block, as an agent for reinforcing the anti-dandruff activity of a composition, especially a cosmetic composition, containing a physiologically acceptable medium and at least one anti-fungal agent selected from among pyridinethione salts, 1-hydroxy-2-pyrrolidone compounds, 2,2′-dithiobis(pyridine N-oxide) and selenium sulfides, and mixtures thereof.

The present invention also features the formulation of at least one block copolymer exclusively containing hydrophilic blocks and comprising at least one polyionic block into anti-dandruff compositions containing a physiologically acceptable medium and at least one anti-fungal agent selected from among pyridinethione salts, 1-hydroxy-2-pyrrolidone derivatives, 2,2′-dithiobis(pyridine N-oxide) and selenium sulfides, and mixtures thereof.

This invention also features a cosmetic hair treatment regime or regimen comprising the application of a cosmetic composition as defined above onto human hair and/or the human scalp. This treatment has the characteristics of a cosmetic process insofar as it makes it possible to improve the aesthetics of the hair and the scalp by imparting to them an improved appearance. In addition, same may be used daily for several months, without medical prescription.

### DETAILED DESCRIPTION OF BEST MODE AND SPECIFIC/PREFERRED EMBODIMENTS OF THE INVENTION

For the purposes of the invention, the term “at least one” means one or more; “at least two” means two or more; etc.

As pyridinethione salts that may be used in the invention, mention may be made of the calcium, magnesium, barium, strontium, zinc or zirconium salts. The zinc salt is particularly preferred.

As a 1-hydroxy-2-pyrrolidone derivative that may be used in the invention, mention may be made of 1-hydroxy4-methyl-6-(2,4,4-trimethylpentyl)-2 (1H)-pyridinone (or piroctone) and piroctone olamine (or octopirox).

As selenium sulfides that may be used in the invention, mention may be made of selenium disulfide and selenium polysulfides of formula SeₓS_{y} in which x+y=8.

Zinc pyridinethione, octopirox or selenium disulfide, or mixtures thereof, are used in particular as anti-fungal agent.

The term “block copolymer” means a polymer resulting from the polymerization of at least two different monomers, each block comprising at least 3 identical repeating or recurring structural units and in general at least 10 identical repeating units.

The term “block copolymer exclusively containing hydrophilic blocks” means a block copolymer for which all the blocks are hydrophilic.

The term “hydrophilic block” means a block whose corresponding homopolymer is soluble or dispersible in water to an amount of at least 1% by weight relative to the (water+homopolymer) assembly, at room temperature (20-25° C.) and atmospheric pressure (about 10⁵ Pa). This homopolymer may be introduced in unmodified form into water or may be neutralized. The (water+homopolymer) assembly has a transparent or translucent appearance to the naked eye. In particular, each hydrophilic block comprises more than 50 mol % of hydrophilic monomer relative to the total number of moles of the block.

The term “polyionic block” means a block comprising at least 2 charges, these charges possibly being of the same sign or of opposite signs.

According to the invention, the block copolymer(s) used comprise at least 2 blocks and for example 2 or 3 blocks. This or these copolymer(s) may also be arranged in star form.

According to the invention, the copolymers containing polyionic hydrophilic blocks may be selected from among: water-soluble or water-dispersed copolymers containing linear or branched blocks, containing a) at least one polyionic macromolecular block and b) at least one nonionic macromolecular block predominantly containing one or more hydrophilic monomers; grafted copolymers.

The copolymers containing polyionic hydrophilic blocks that may be used in the invention are manufactured according to the processes known to those skilled in the art by block copolymerization of at least 2 monomers, at least one of which is selected from among ionic hydrophilic monomers, for instance anionic hydrophilic monomers, cationic hydrophilic monomers and/or amphoteric hydrophilic monomers, and optionally of at least one nonionic hydrophilic monomer. The block copolymers of the invention may be prepared via the synthetic processes conventionally used for obtaining block polymers. Examples that may be mentioned include anionic or cationic polymerization, and controlled free-radical polymerization (see “New Method of Polymer Synthesis”, Blackie Academic & Professional, London, 1995, volume 2, page 1, or Trends Polym. Sci., 4, page 183 (1996) from C. J. Hawker), which may be performed according to various processes, for instance atom transfer radical polymerization (or ATRP) (see JACS, 117, page 5614 (1995) from Matyjasezwski et al.), the method of free radicals such as nitroxides (Georges et al., Macromolecules, 1993, 26, 2987).

The number-average molecular mass of each block, whether it is in the form of a copolymer or a homopolymer, is preferably within the range from 500 to 100,000, in particular from 800 to 50,000 and better still from 1,500 to 40,000.

Among the anionic hydrophilic monomers included in the constitution of the block copolymers of the invention, mention may be made especially of:
ethylenically unsaturated monomers comprising at least one carboxylic acid, phosphoric acid or sulfonic acid function, for instance acrylic acid, methacrylic acid, 1,4-phenylenediacrylic acid, crotonic acid, itaconic acid, fumaric acid, maleic acid, citraconic acid, diacrylic acid, coumaric acid, dimethylfumaric acid, mesaconic acid, muconic acid, acrylamidopropanesulfonic acid, 2-acrylamido-2-methylpropanesulfonic acid, 4-styrenesulfonic acid, vinylbenzoic acid, vinylphosphoric acid, vinylbenzenesulfonic acid, 2-propene-1-sulfonic acid, vinylsulfonic acid, vinylphosphonic acid, vinylglycolic acid, ethylidenepropionic acid, ethylidenesuccinic acid, glutaconic acid, diallylmalonic acid, and cinnamic acid and its hydroxylated and/or methoxylated derivatives;
carboxylic anhydrides bearing a vinyl bond, such as maleic anhydride or citraconic anhydride;
2-carboxyethyl (meth)acrylate, sulfopropyl methacrylate or acrylate (CH₂═C(CH₃)CO₂(CH₂)₃SO₃H), diallyl maleate (C₃H₅—CO₂—CH═CH—CO₂—C₃H₅), sulfoethyl methacrylate or acrylate, and vinyl methyl sulfone;
and the salts thereof;
combinations thereof.

In particular, the anionic hydrophilic monomer comprises a carboxylic function and is selected from among acrylic acid, 1,4-phenylenediacrylic acid, citraconic acid or citraconic anhydride, cinnamic acid and its hydroxylated and/or methoxylated derivatives, glutaconic acid, itaconic acid, methacrylic acid, maleic acid or anhydride, mesaconic acid, muconic acid and hydromuconic acid.

The neutralization of the anionic groups may be performed via a mineral base such as LiOH, NaOH, KOH, Ca(OH)₂, NH₄OH or Zn(OH)₂; or via an organic base such as a primary, secondary or tertiary alkylamine, especially triethylamine, monoethylamine or butylamine. This primary, secondary or tertiary alkylamine may comprise one or more nitrogen and/or oxygen atoms and may thus comprise, for example, one or more hydroxyl functions; mention may be made especially of amino-2-methyl-2-propanol, triethanolamine and dimethylamino-2-propanol. Mention may also be made of lysine or 3-(dimethylamino)propylamine.

Among the cationic hydrophilic monomers included in the constitution of the block copolymers of the invention, mention may be made of ethylenically unsaturated monomers comprising at least one primary, secondary or tertiary amine function, for instance:
2-vinylpyridine, 4-vinylpyridine or N-(C₁-C₆)alkylpiperidines;
aminoalkyl (meth)acrylates such as N,N-di(C₁-C₄)alkylamino(C₁-C₆)alkyl (meth)acrylates or (C₁-C₄)alkylamino(C₁-C₄)alkyl (meth)acrylates and especially N,N-dimethylaminoethyl (meth)acrylate, N,N-diethylaminoethyl (meth)acrylate, 2-aminoethyl (meth)acrylate or 2-(tert-butylamino)ethyl (meth)acrylate;
aminoalkyl(meth)acrylamides such as N,N-di(C₁-C₄)alkylamino(C₁-C₆)alkyl(meth)acrylamides or (C₁-C₄)alkylamino(C₁-C₆)alkyl(meth)acrylamides, and especially N,N-dimethylaminopropyl(meth)acrylamide, N,N-dimethylaminoethyl(meth)acrylamide, 3-aminopropyl(meth)acrylamide and acecainide;
vinylamine, allylamine, vinylimidazole, 2-(diethylamino)ethylstyrene and (2-(diethylamino)ethyl)-2-(2-propenyloxy)benzamide;
and also salts thereof and quaternized forms thereof;
combinations thereof.

Among the salified forms of the cationic monomers that may be mentioned are the salts of mineral acids such as sulfuric acid, hydrochloric acid, hydrobromic acid, hydriodic acid, phosphoric acid and boric acid. Mention may also be made of the salts of organic acids, which may comprise one or more carboxylic, sulfonic or phosphonic acid groups. They may be linear, branched or cyclic aliphatic acids, or alternatively aromatic acids. These acids may also comprise one or more hetero atoms selected from O and N, for example in the form of hydroxyl groups. Mention may be made especially of propionic acid, acetic acid, terephthalic acid, citric acid, oxalic acid and tartaric acid.

The quaternized forms of the cationic monomers may be obtained by reaction with compounds containing labile halogen, in particular such as alkyl halides such as C₁-C₁₂ alkyl chlorides or bromides, for example methyl bromide or ethyl chloride; or alternatively with sodium chloroacetate. The quaternized forms may also be obtained by reaction with cyclic sulfones, for example propane sulfone.

Among the amphoteric hydrophilic monomers included in the constitution of the block copolymers of the invention, mention may be made of monomers comprising at least one basic nitrogen atom and at least one carboxylic, phosphoric or sulfonic group, in particular such as cationic monomers comprising a primary, secondary, tertiary or quaternary amine function and a carboxylic, phosphoric or sulfonic function, or alternatively zwitterionic monomers of carboxybetaines or of sulfobetaines.

As examples of amphoteric monomers that may be used in the invention, mention may be made of
the zwitterionic monomers of formula (I) below: 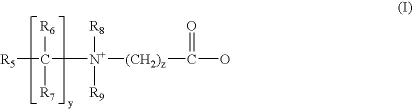
   in which R₅ denotes an unsaturated polymerizable group such as an acrylate, methacrylate, acrylamide or methacrylamide group, y and z represent an integer from 1 to 3, R₆ and R₇ represent a hydrogen atom, methyl, ethyl or propyl, R₈ and R₉ represent a hydrogen atom or an alkyl radical such that the sum of the carbon atoms in R₈ and R₉ does not exceed 10;
aminocinnamic acid and amino acids such as arginine;
monomers corresponding to formula (II) below: 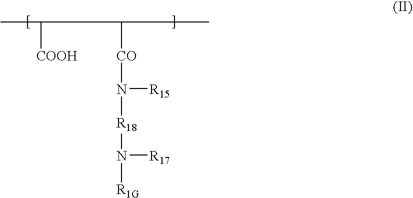
   in which R₁₄ represents a hydrogen atom or a CH₃O, CH₃CH₂O or phenyl radical, R₁₅ and R₁₆ independently denote hydrogen or a lower C₁ to C₆ alkyl radical such as methyl or ethyl, R₁₇ denotes a lower C₁ to C₆ alkyl radical such as methyl, ethyl or a radical corresponding to the formula: —R₁₈—N(R₁₆)₂, R₁₈ representing a —CH₂—CH₂—, —CH₂—CH₂—CH₂— or —CH₂—CH(CH₃)— group, R₁₆ having the meanings mentioned above,
maleic anhydride partially modified by semiamidation with an N,N-dialkylaminoalkylamine such as N,N-dimethylaminopropylamine or by semiesterification with an N,N-dialkanolamine;
mixtures thereof.

Among the nonionic hydrophilic monomers included in the constitution of the block copolymers of the invention, mention may be made, inter alia, of acrylamide, methacrylamide, C₁₋₆ N-alkyl or C₁₋₃ N,N-dialkyl acrylamides, polyethylene glycol acrylate, polyethylene glycol methacrylate, N-vinylacetamide, N-methyl-N-vinylacetamide, N-vinylformamide, N-methyl-N-vinylformamide, N-vinyllactams comprising a cyclic group of 4 to 9 carbon atoms, vinyl alcohol (copolymerized in the form of vinyl acetate and then hydrolysed), ethylene oxide and/or propylene oxide, hydroxyethyl acrylate, vinylpyrrolidone, vinyltriazole, hydroxypropyl acrylate, hydroxyethyl methacrylate, hydroxypropyl methacrylate, amino acids, methyl-2-oxazoline or ethyl-2-oxazoline.

The copolymers that may be used in the invention are in particular those described in WO-A-03/050184, WO-A-03/050185 and WO-03/050195 to Rhodia.

Preferably, linear block copolymers are used, and in particular diblock copolymers containing linear blocks.

More preferably, the block copolymers according to the invention comprise at least one polyionic hydrophilic block and at least one nonionic hydrophilic block.

According to one particular embodiment of the invention, the block copolymer(s) that may be used in the invention is(are) selected from among the polymers of formulae N—P, (P—N)ₙ—P, N—(P—N)ₙ and N—(P—N)ₙ—P, in which N is a nonionic hydrophilic block, P is a polyionic hydrophilic block and n is an integer ranging from 1 to 5,000. The blocks (P—N)ₙ of these copolymers may be of the same length or of different length.

According to one particular embodiment of the invention, the copolymer of the invention comprises a nonionic hydrophilic block and a polycationic hydrophilic block.

Advantageously, the block copolymer(s) that may be used in the invention comprise(s) one or more polyacrylic acid, poly(meth)acrylic acid blocks or a salt thereof or an amide thereof and one or more aminoalkyl (meth)acrylate blocks or a salt thereof or a quaternized form thereof. Examples of these blocks that may be mentioned include the poly(acrylamide) block and the poly(trimethylammonium ethylacrylate methosulfate) block.

The compositions according to the invention may comprise one or more anti-fungal agents mentioned previously. Their amount depends on the nature of the physiological medium used and also on the amount of hydrophilic block copolymer used and on the desired level of anti-dandruff activity. In practice, the anti-fungal agent or the mixture of anti-fungal agents may represent from 0.001% to 10% by weight and preferably from 0.1% to 5% by weight relative to the total weight of the composition.

The compositions according to the invention may contain one or more block copolymers mentioned previously. Their amount also depends on the nature of the physiological medium used and also on the amount of anti-fungal agent used and on the desired level of anti-dandruff activity. In practice, the block copolymer or the mixture of block copolymers may represent from 0.01% to 20% by weight and preferably from 0.05% to 10% by weight relative to the total weight of the composition.

The compositions according to the invention also contain a physiologically acceptable medium. The term “physiologically acceptable medium” means a non-toxic medium that may be applied to the human scalp and the hair. For the purposes of the invention, the term “cosmetic” means a composition that may be sold without medical prescription.

In particular, this medium contains an aqueous phase containing water and optionally at least one water-miscible organic solvent, for instance C₂-C₆ monoalcohols such as ethanol, isopropanol or n-butanol, polyols, for instance propylene glycol or glycerol, or glycol ethers. This medium may contain an oily phase containing one or more fatty substances that are liquid at room temperature (25° C.) and atmospheric pressure, and which are water-immiscible, known as “oils”.

The physiologically acceptable medium may also comprise at least one surfactant selected from among nonionic surfactants, anionic surfactants, cationic surfactants and amphoteric surfactants, and mixtures thereof.

The compositions according to the invention, after application to human hair and the scalp, may or may not be rinsed off with water or by shampooing. It may be in any form conventionally used in the field under consideration, for example in the form of a more or less thickened lotion, a gel, a cream, a spray or a mousse. This composition may be a monophase or multiphase composition. It may also be, for example, in the form of a shampoo, a conditioner or a treating lotion for the hair and the scalp. The composition of the invention is advantageously in the form of a shampoo.

Thus, the present invention also features a hair shampoo containing a physiologically acceptable medium, a washing base, at least one anti-fungal agent and at least one block copolymer exclusively containing hydrophilic blocks as defined above.

The compositions according to the invention may also contain as washing base at least one surfactant selected from among anionic, nonionic and amphoteric surfactants, and mixtures thereof.

As anionic surfactants that may be used in the present invention, mention may be made especially of salts, in particular alkali metal salts such as sodium salts, ammonium salts, amine salts, amino alcohol salts or alkaline-earth metal salts, for example magnesium salts, of the following types: alkyl sulfates, alkyl ether sulfates, alkylamido ether sulfates, alkylarylpolyether sulfates, monoglyceride sulfates; alkyl sulfonates, alkylamide sulfonates, alkylaryl sulfonates, α-olefin sulfonates, paraffin sulfonates, alkyl sulfosuccinates, alkyl ether sulfosuccinates, alkylamide sulfosuccinates, alkyl sulfoacetates, acyl sarcosinates and acyl glutamates, the alkyl and acyl groups of all these compounds containing from 6 to 24 carbon atoms, and the aryl group preferably denoting a phenyl or benzyl group, and mixtures thereof.

It is also possible to use as anionic surfactant monoesters of C₆₋₂₄ alkyl and of polyglycoside-dicarboxylic acids, such as alkyl glucoside citrates, polyalkyl glycoside tartrates and polyalkyl glycoside sulfosuccinates, alkyl sulfosuccinamates, acyl isethionates and N-acyltaurates, the alkyl or acyl group of all these compounds containing from 12 to 20 carbon atoms, and mixtures thereof.

Another group of anionic surfactants that may be used in the compositions of the present invention is that of acyl lactylates in which the acyl group contains from 8 to 20 carbon atoms.

In addition, mention may also be made of alkyl-D-galactosiduronic acids and salts thereof, and also polyoxyalkylenated (C₆₋₂₄ alkyl)ether-carboxylic acids, polyoxyalkylenated (C₆₋₂₄ alkyl)(C₆₋₂₄ aryl)ether-carboxylic acids and polyoxyalkylenated (C₆₋₂₄ alkyl)amidoether-carboxylic acids and salts thereof, in particular those comprising from 2 to 50 ethylene oxide units, and mixtures thereof.

Alkyl sulfates, alkyl ether sulfates and alkyl ether carboxylates, and mixtures thereof, are preferably used as anionic surfactant, in particular in the form of alkali metal, alkaline-earth metal, ammonium, amine or amino alcohol salts.

As amphoteric surfactants that may be used in the present invention, mention may especially be made of aliphatic secondary or tertiary amine derivatives in which the aliphatic group is a linear or branched chain containing from 8 to 22 carbon atoms and containing at least one anionic group, for example carboxylate, sulfonate, sulfate, phosphate or phosphonate. Mention may also be made of (C₈-C₂₀)alkylbetaines, sulfobetaines, (C₈-C₂₀)alkylamido(C₆-C₈)alkylbetaines or (C₈-C₂₀)alkylamido(C₆-C₈)alkylsulfobetaines, and mixtures thereof.

Among the amine derivatives, mention may be made of the products sold under the name Miranol®, as described in U.S. Pat. Nos. 2,528,378 and 2,781,354 and classified in the CTFA dictionary, 3rd edition, 1982, under the names Amphocarboxyglycinate and Amphocarboxypropionate, having the respective structures (1) and (2):
Ra—CONHCH₂CH₂—N(R_{b})(R_{c})(CH₂COO⁻) (1)
in which:
Ra represents an alkyl group derived from an acid Ra-COOH present in hydrolysed coconut oil, a heptyl, nonyl or undecyl group,
Rb represents a β-hydroxyethyl group, and
Rc represents a carboxymethyl group; and
   Ra′—CONHCH₂CH₂—N(B)(C) (2)
   in which:
B represents —CH₂CH₂OX′,
C represents —(CH₂)_{z}—Y′, with z=1 or 2,
X′ represents a —CH₂CH₂—COOH group or a hydrogen atom,
Y′ represents —COOH or a —CH₂—CHOH—SO₃H group,
Rₐ′ represents an alkyl group of an acid Rₐ′—COOH present in coconut oil or in hydrolysed linseed oil, an alkyl group, in particular a C₁₇ alkyl group and its iso form, an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

By way of example, mention may be made of the cocoamphodiacetate sold by Rhodia under the trademark Miranol® C2M concentrate.

Among the amphoteric surfactants that are preferably used are (C₈₋₂₀ alkyl)betaines, (C₈₋₂₀ alkyl)amido(C₆₋₈ alkyl)betaines and alkylamphodiacetates, and mixtures thereof.

As nonionic surfactants that may be used in the compositions according to the invention, mention may be made of the known compounds described especially in the book “Handbook of Surfactants” by M. R. Porter, published by Blackie & Son (Glasgow and London), 1991, pp. 116-178). They are selected in particular from among polyethoxylated, polypropoxylated or polyglycerolated fatty acids, (C₁-C₂₀)alkylphenols, α-diols or alcohols having a fatty chain containing, for example, 8 to 18 carbon atoms, it being possible for the number of ethylene oxide or propylene oxide groups to range in particular from 2 to 50 and for the number of glycerol groups to range in particular from 2 to 30, and mixtures thereof.

As nonionic surfactants that may be used in the invention, mention may also be made of condensates of ethylene oxide and of propylene oxide with fatty alcohols; polyethoxylated fatty amides preferably having from 2 to 30 ethylene oxide units, polyglycerolated fatty amides containing on average 1 to 5, and in particular 1.5 to 4, glycerol groups; ethoxylated fatty acid esters of sorbitan having from 2 to 30 ethylene oxide units; fatty acid esters of sucrose; fatty acid esters of polyethylene glycol; (C₆₋₂₄ alkyl)polyglycosides; N—(C₆₋₂₄ alkyl)glucamine derivatives; amine oxides such as (C₁₀-C₁₄)alkylamine oxides or N—(C₁₀₋₁₄ acyl)aminopropylmorpholine oxides; and mixtures thereof.

Among the nonionic surfactants mentioned above, (C₆₋₂₄ alkyl)polyglycosides are preferably used.

According to one particular embodiment of the invention, the washing base contains at least one anionic surfactant and at least one amphoteric or nonionic surfactant.

The total amount of surfactant is generally within the range from 0.01% to 50% by weight and preferably from 0.1% to 25% by weight relative to the total weight of the composition.

In particular, when the composition of the invention is in the form of a shampoo, the total amount of surfactants (or washing base) is chosen in particular from 4% to 50% by weight, for example from 8% to 25% by weight, relative to the total weight of the cosmetic composition.

The compositions according to the invention may also contain at least one additional ingredient conventionally used in the fields under consideration and chosen from cosmetic active principles with a beneficial effect on the skin and/or on the hair, for instance the zinc salts of an organic acid (acetate, glycolate, lactate, gluconate or citrate) or of a mineral acid (chloride and sulfate), vitamins (E, C, B₂, B₅ and F), UV-screening agents, free-radical scavengers, preservatives, ceramides, plant extracts and formulation additives such as anionic, nonionic, cationic or amphoteric film-forming polymers, different from the polyionic hydrophilic block copolymers described above, aqueous-phase or oily-phase polymeric thickeners, aqueous-phase non-polymeric thickeners, for instance hydroxylated or non-hydroxylated fatty acid salts or amides, nacreous agents, opacifiers, dyes that are soluble in the medium, pigments, fillers, fragrances, oils of mineral, plant and/or synthetic origin, esters of fatty acids and/or of fatty alcohols, waxes, pH stabilizers, for instance acids, bases or salts, organic solvents, silicones and electrolytes, and mixtures thereof.

The amounts of the various additional ingredients of the composition according to the invention are those generally used in the fields under consideration and are in particular within the range from 0.001% to 20% of the total weight of the composition. In addition, this composition is prepared according to the usual methods.

Needless to say, one skilled in the art will take care to select the optional additional ingredients and/or the amount thereof such that the advantageous properties of the composition according to the invention, i.e., the anti-dandruff activity, are not, or are not substantially, adversely affected by the envisaged addition.

Advantageously, the pH of the compositions of the present invention is in the range from 2 to 11 and preferably from 3 to 10, for example from 5 to 8.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

### Examples 1 and 2

The efficacy of two anti-fungal agents, zinc pyridinethione (Zn omadine manufactured or sold by Olin) and piroctone olamine (octopirox manufactured or sold by Hoechst) at a concentration of 1% Active Material (A.M.) in a surfactant washing base containing a sodium lauryl ether (2.2 mol) sulfate at 10% A.M. and of cocopropylbetaine at 5% A.M. in the presence or absence of 1% A.M. of a poly(trimethylammonium ethylacrylate methosulfate)-b-poly(acrylamide) diblock copolymer (weight-average molecular weight of 11,000/30,000) was determined. The rest of the composition contains a quantity of water sufficient to make 100%.

The test used to compare the efficacy of the compositions according to the invention relative to compositions free of hydrophilic block copolymer is the standard Minimum Inhibitory Concentration (MIC) method described especially in EP-A-1,048,288.

The presence of the diblock copolymer very markedly improves the anti-dandruff efficacy of the anti-fungal agents under consideration.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. A topically applicable cosmetic/dermatological composition comprising at least one anti-fungal agent selected from the group consisting of pyridinethione salts, 1-hydroxy-2-pyrrolidone compounds, 2,2′-dithiobis(pyridine N-oxide), selenium sulfides, and mixtures thereof, and at least one wholly hydrophilic block copolymer which comprises at least one polyionic block, formulated into a topically applicable, physiologically acceptable medium therefor.

2. The cosmetic/dermatological composition as defined by claim 1, comprising at least one calcium, magnesium, barium, strontium, zinc or zirconium pyridinethione salt.

3. The cosmetic/dermatological composition as defined by claim 1, comprising at least one 1-hydroxy-2-pyrrolidone compound selected from piroctone or piroctone olamine.

4. The cosmetic/dermatological composition as defined by claim 1, comprising at least one anti-fungal agent being selected from the group consisting of zinc pyridinethione, octopirox and selenium disulfide, and mixtures thereof.

5. The cosmetic/dermatological composition as defined by claim 1, comprising at least one wholly hydrophilic linear block copolymer.

6. The cosmetic/dermatological composition as defined by claim 1, comprising at least one wholly hydrophilic diblock copolymer containing linear blocks.

7. The cosmetic/dermatological composition as defined by claim 1, each block of said at least one wholly hydrophilic block copolymer having a number-average molecular mass ranging from 500 to 100,000.

8. The cosmetic/dermatological composition as defined by claim 1, comprising at least one wholly hydrophilic block copolymer comprising at least one nonionic hydrophilic block and at least one polyionic hydrophilic block.

9. The cosmetic/dermatological composition as defined by claim 1, comprising at least one wholly hydrophilic block copolymer comprising a polymer of formulae N—P, (P—N)ₙ—P, N—(P—N)ₙ or N—(P—N)ₙ—P, in which N is a nonionic hydrophilic block, P is a polyionic hydrophilic block and n is an integer ranging from 1 to 5,000.

10. The cosmetic/dermatological composition as defined by claim 1, said at least one wholly hydrophilic block copolymer comprising a nonionic hydrophilic block and a polycationic hydrophilic block.

11. The cosmetic/dermatological composition as defined by claim 1, said at least one wholly hydrophilic block copolymer comprising one or more polyacrylic acid or poly(meth)acrylic acid blocks or salt or amide thereof and one or more aminoalkyl (meth)acrylate blocks or salt or quaternized form thereof.

12. The cosmetic/dermatological composition as defined by claim 1, said at least one anti-fungal agent comprising from 0.001% to 10% thereof.

13. The cosmetic/dermatological composition as defined by claim 1, said at least one wholly hydrophilic block copolymer comprising from 0.01% to 20% by weight thereof.

14. The cosmetic/dermatological composition as defined by claim 1, said physiologically acceptable medium comprising water and optionally at least one water-miscible organic solvent.

15. The cosmetic/dermatological composition as defined by claim 1, formulated as a lotion, a gel, a cream, a spray or a mousse.

16. The cosmetic/dermatological composition as defined by claim 1, formulated in monophase or multiphase form.

17. The cosmetic/dermatological composition as defined by claim 1, said physiologically acceptable medium comprising at least one surfactant selected from the group consisting of nonionic surfactants, anionic surfactants, cationic surfactants and amphoteric surfactants, and mixtures thereof.

18. The cosmetic/dermatological composition as defined by claim 17, comprising from 0.01% to 50% by weight of surfactant.

19. The cosmetic/dermatological composition as defined by claim 1, comprising a washing base.

20. The cosmetic/dermatological composition as defined by claim 19, said washing base comprising at least one surfactant selected from the group consisting of anionic, nonionic and amphoteric surfactants, and mixtures thereof.

21. The cosmetic/dermatological composition as defined by claim 19, said washing base comprising at least one anionic surfactant and at least one amphoteric or nonionic surfactant.

22. The cosmetic/dermatological composition as defined by claim 19, said washing base comprising from 4% to 50% by weight thereof.

23. The cosmetic/dermatological composition as defined by claim 1, comprising at least one additional ingredient selected from the group consisting of cosmetic active principles and formulation additives.

24. A hair shampoo comprising a washing base, at least one anti-fungal agent selected from the group consisting of pyridinethione salts, 1-hydroxy-2-pyrrolidone compounds, 2,2′-dithiobis(pyridine N-oxide) and selenium sulfides, and mixtures thereof, and at least one wholly hydrophilic block copolymer, formulated into a topically applicable, physiologically acceptable medium therefor.

25. A regime or regimen for the treatment of dandruff and/or squamae of the scalp, comprising topically applying onto the hair and/or scalp of an individual in need of such treatment, a thus effective amount of a cosmetic/dermatological composition which comprises at least one anti-fungal agent selected from the group consisting of pyridinethione salts, 1-hydroxy-2-pyrrolidone compounds, 2,2′-dithiobis(pyridine N-oxide) and selenium sulfides, and mixtures thereof, and at least one wholly hydrophilic block copolymer which comprises at least one polyionic block, formulated into a topically applicable, physiologically acceptable medium therefor.
